(19) United States
(12) Patent Application Publication
(10) Pub. No.: US 2024/0189317 A1
(43) Pub. Date: Jun. 13, 2024

(54) ANTIVIRAL COMPOSITIONS AND METHODS

(71) Applicant: AI Therapeutics, Inc., Guilford, CT (US)

(72) Inventors: Peter R. Young, Guilford, CT (US); Sean Landrette, Guilford, CT (US); Carlo Spirli, Guilford, CT (US); Keith Fandrick, Guilford, CT (US); Murat Gunel, Guilford, CT (US)

(21) Appl. No.: 18/281,942

(22) PCT Filed: Mar. 15, 2022
(86) PCT No.: PCT/US2022/020326

§ 371 Date: Sep. 13, 2023

Related U.S. Application Data
(60) Provisional application No. 63/161,663, filed on Mar. 16, 2021.

(51) Int. Cl. A61K 31/5377; A61K 31/245; A61P 31/12

(52) U.S. Cl. CPC A61K 31/5377; A61K 31/245; A61P 31/12

(57) ABSTRACT

The present invention relates to compositions and related methods of using a PIKfyve inhibitor, such as apilimod, in combination with a serine protease inhibitor, for treating or preventing viral disease.

### RELATED APPLICATIONS

This application claims priority to and benefit of U.S. Provisional Application No. 63/161,663, filed on Mar. 16, 2021; the contents of which are hereby incorporated in its entirety.

### FIELD OF THE INVENTION

The present invention relates to compositions and methods for treating or preventing viral disease.

### BACKGROUND OF THE INVENTION

Many viruses enter the cell via endocytosis and utilize the endosomal network as a means to infiltrate the cell and replicate. For example, viral entry into cells may be mediated by a viral glycoprotein (GP), which attaches viral particles to the cell surface, delivers them to endosomes, and catalyzes fusion between viral and endosomal membranes. Rab9 GTPase was shown to be required for replication of HIV-1, filoviruses (such as Ebola and Marburg), and measles virus. (Murray et al. 2005 J. Virology 79:11742-11751). Silencing Rab9 expression dramatically inhibited HIV replication, as did silencing the host genes encoding TIP47, p40, and PIKfyve, which also facilitate late-endosome-to-trans-Golgi vesicular transport. Reducing Rab9 expression also inhibited the replication of the enveloped Ebola and Marburg filoviruses and that of measles virus, but not the non-enveloped reoviruses. Murray et al. 2005 J. Virology 79:11742-11751

Coronaviruses are enveloped RNA viruses that cause respiratory, hepatic, and neurological disease (Weiss et al. (2011) Adv Virus Res 81:85-164; Cui et al., 2019 Nat Rev Microbiology 17:181-192). Recent outbreaks of severe acute respiratory syndrome (SARS) and Middle East respiratory syndrome (MERS) have revealed the potential for high pathogenicity (Cui et al., (2019) Nat Rev Microbiology 17:181-192). The high prevalence, wide distribution, genetic diversity, recombination, and frequent cross species infections of coronaviruses lend to the emergence of novel pathogenic strains (Cui et al., (2019) Nat Rev Microbiology 17:181-192; Wong et al., (2015) Cell Host and Microbe 18(4):398-401). Indeed, a novel pathogenic coronavirus, SARS-CoV-2, causing pneumonia and high mortality emerged in 2019 and resulted in a global pandemic (Zhu et al., 2020, N Engl J Med 382:727-733; Huang et al. (2020); Lancet Infect Dis, 2020:1010-1011). The resulting disease has been named COVID-19.

Attempts to treat SARS and MERS with approved antivirals and immunomodulators have proven largely ineffective in early clinical trials (Zumla et al., (2016), Nat Rev Drug Discov. 15(5):327-47). More recently some progress has been made in the identification of vaccines to prevent infection, but the administration of these to a substantial proportion of the world population will take some time, during which the virus will continue to circulate, causing morbidity and mortality, as well as leading to the development of mutated forms of the virus with increased potency, infectivity and potential for resistance to currently approved vaccines and therapies (Na et al. (2021) J Microbiol 59:332-340). It is also not known how long is the protection that these vaccines provide, and none will prevent infection by newly emerging coronaviruses. Hence, there is a need for prophylactic and therapeutic treatments that lessen disease burden, reduce symptoms and lower the rate of hospitalization and death. Some existing drugs have shown promise in treating hospitalized patients and have received approval from regulatory agencies including remdesivir, baracitinib and dexamethasone but there is an unmet need for outpatient treatments (Khani et al. (2021) J Clin Pharmacol January. doi 10.1002/jpch.1822). Thus, the treatment and prophylaxis of coronavirus infection remains an unmet clinical need.

The present invention addresses the need for new therapies for treating and preventing viral infections, particularly coronavirus infections.

### SUMMARY OF THE INVENTION

The present invention provides compositions and methods related to the use of PIKfyve inhibitors in combination with at least one serine protease inhibitor for treatment and prophylaxis of coronavirus infections in a subject, preferably a human subject, in need of such treatment or prevention.

Accordingly, the invention provides a pharmaceutical composition comprising apilimod, or a pharmaceutically acceptable salt thereof, and a serine protease inhibitor. In embodiments, the apilimod is in the form of its free base. In embodiments, the apilimod is in the form of a salt, and the pharmaceutically acceptable salt is a monosalt selected from the group consisting of chloride, fumarate, glycolate, D or L-lactate, maleate, malonate, mesylate, 1-hydroxy-2-naphthoate, phosphate, succinate, and L-tartrate; or a disalt selected from the group consisting of mesylate, chloride, and bromide. In embodiments, the apilimod is in the form of a dimesylate salt.

In embodiments of the pharmaceutical compositions described here, the serine protease inhibitor is selected from the group consisting of actinonin, amiloride, apixaban, aprotinin, argatroban, aspartame, atropine, batimastat, betrixaban, bromhixine, bupivacaine, camostat, cefixime, creatinine, dabigatran, dansyl-D-phenylalanine, darapladib, darexaban, dizocilpine, doxorubicin, doxycycline, ecopladib, edoxaban, efipladib, epichlortetracycline, epidemeclocycline, eribaxaban, gabexate, ginsenoside, giripladib, ilomastat, letaxaban, leupeptin, luteolin, marimastat, nafamostat, neostigmine, noradrenaline, otamixaban, phosphoramidon, prinomastat, rivaroxaban, sepimostat, sivelestat, suramin, tanomastat, thiorphan, tranexamic acid, trocade, tropicamide, varespladib, variabilin, and ximelagatran. In embodiments, the serine protease inhibitor is selected from the group consisting of aprotinin, bromhixine, camostat, gabexate, nafamostat, otamixaban, and sepimostat.

In embodiments of the pharmaceutical compositions described here, the serine protease inhibitor is an inhibitor of transmembrane protease, serine 2 (TMPRSS2). In embodiments, the TMPRSS2 inhibitor is selected from the group consisting of aprotinin, bromhixine, camostat, marimastat, nafamostat, phosphoramidon, sepimostat, and tannic acid.

In embodiments of the pharmaceutical compositions described here, the serine protease inhibitor is a TMPRSS2 inhibitor selected from the group consisting of camostat, nafamostat, and sepimostat. In embodiments of the pharmaceutical compositions described here, the serine protease inhibitor is camostat or nafamostat.

In embodiments of the pharmaceutical compositions described here, the composition is in the form of an oral dosage form or an intravenous dosage form.

The invention also provides methods for treating or preventing viral disease in a subject in need thereof, the method comprising administering to the subject a pharmaceutical composition comprising apilimod, or a pharmaceutically acceptable salt thereof, and a serine protease inhibitor. In embodiments, the apilimod is in the form of a free base. In embodiments, the apilimod is in the form of a salt and the pharmaceutically acceptable salt is a monosalt selected from the group consisting of chloride, fumarate, glycolate, D or L-lactate, maleate, malonate, mesylate, 1-hydroxy-2-naphthoate, phosphate, succinate, and L-tartrate; or a disalt selected from the group consisting of mesylate, chloride, and bromide. In embodiments, the apilimod is in the form of a dimesylate salt.

In embodiments of the methods described here, the serine protease inhibitor is selected from the group consisting of actinonin, amiloride, apixaban, aprotinin, argatroban, aspartame, atropine, batimastat, betrixaban, bromhixine, bupivacaine, camostat, cefixime, creatinine, dabigatran, dansyl-D-phenylalanine, darapladib, darexaban, dizocilpine, doxorubicin, doxycycline, ecopladib, edoxaban, efipladib, epichlortetracycline, epidemeclocycline, eribaxaban, gabexate, ginsenoside, giripladib, ilomastat, letaxaban, leupeptin, luteolin, marimastat, nafamostat, neostigmine, noradrenaline, otamixaban, phosphoramidon, prinomastat, rivaroxaban, sepimostat, sivelestat, suramin, tanomastat, thiorphan, tranexamic acid, trocade, tropicamide, varespladib, variabilin, and ximelagatran. In embodiments, the serine protease inhibitor is selected from the group consisting of aprotinin, bromhixine, camostat, gabexate, nafamostat, otamixaban, and sepimostat.

In embodiments of the methods described here, the serine protease inhibitor is an inhibitor of transmembrane protease, serine 2 (TMPRSS2). The method of claim 3, wherein the serine protease inhibitor is selected from camostat or nafamostat. In embodiments, the TMPRSS2 inhibitor is selected from the group consisting of aprotinin, bromhixine, camostat, marimastat, nafamostat, phosphoramidon, sepimostat, and tannic acid. In embodiments, the serine protease inhibitor is a TMPRSS2 inhibitor selected from the group consisting of camostat and nafamostat.

In embodiments of the methods described here, the subject is a human.

In embodiments of the methods described here, the subject in need is one who has symptoms of a viral infection, preferably a respiratory viral infection, including one or more of sore throat, nasal congestion and/or discharge, shortness of breath, difficulty breathing, and fever. In embodiments, the viral disease is caused by a coronavirus. In embodiments, the coronavirus is selected from SARS-CoV-1, MERS-CoV, and SARS-CoV-2. In embodiments, the coronavirus is SARS-CoV-2.

In embodiments of the methods described here, the apilimod is administered in a separate dosage form, or in the same dosage form, as the serine protease inhibitor.

In embodiments of the methods described here, the apilimod and the serine protease inhibitor may be administered as part of an antiviral regimen that may include one or more additional active agents or antiviral agents.

The invention also provides a pharmaceutical pack or kit comprising, in separate containers or in a single container, a unit dose of apilimod and a unit dose of a serine protease inhibitor. In embodiments, the pharmaceutical pack or kit comprises a unit dose of apilimod, or a pharmaceutically acceptable salt thereof, and a unit dose of camostat or nafamostat, in the same or separate containers as the apilimod.

### BRIEF DESCRIPTION OF THE DRAWINGS

FIG. 1 is a line graph showing dose response curves for apilimod (circles) and camostat (squares), each as single agent, on viral titer (and thus infectivity) in Caco-2 cells. Each point is an average of two single experiments for each of the 8 doses of each single agent treatment. Viability (triangles) is also shown.

FIG. 2 is a bar graph showing activity of apilimod, camostat, and an exemplary synergistic combination of apilimod (62.5 nM) and camostat (562.5 nM) (“Combo”), in preventing SARS-CoV-2 infection in Caco-2 cells. *p<0.05; **p<0.01; ***p<0.001.

### DETAILED DESCRIPTION OF THE INVENTION

The present invention is based, in part, on a finding of unexpected synergistic activity of the PIKfyve inhibitor, apilimod, with serine protease inhibitors, particularly inhibitors of the serine protease, transmembrane protease, serine 2 (TMPRSS2), in preventing SARS-CoV-2 infection in vitro.

Emerging evidence indicates that infection of cells by coronaviruses can proceed by two different pathways (Hoffman et al (2020) Cell 181 1-10). One route involves viral endocytosis via endosomes, which traffic to and fuse with the lysosome. Upon entering the acidic lysosome, the viral spike protein is cleaved resulting in a change of conformation of the virus allowing viral insertion into the lysosomal membrane and injection of viral RNA into the cytoplasm. A second pathway involves cleavage of the spike protein by the cellular enzyme TMPRSS2, a serine protease expressed at the cell surface. This cleavage results in direct insertion of viral RNA into the cytoplasm from the plasma membrane. Recently, TMPRSS2 inhibitors have demonstrated an ability to inhibit viral infection (Breining et al. (2021) Basic Clin Pharmacol Toxicol 128:204-212; Hoffman et al. (2020) Antimicrobial Agents and Chemotherapy 64:e00754-20).

The present invention is based, in part, on a finding of unexpected synergistic activity between the PIKfyve inhibitor, apilimod, and serine protease inhibitors, particularly inhibitors of TMPRSS2, in preventing SARS-CoV-2 infection in vitro. Accordingly, the present invention provides compositions and methods related to the use of a PIKfyve inhibitor in combination with a serine protease inhibitor for treating or preventing a coronavirus infection in a subject, preferably a human subject, in need of such treatment or prevention.

In accordance with any of the embodiments described here, the coronavirus infection may be caused by a coronavirus selected from the group consisting of severe acute respiratory system virus (SARS-CoV), SARS-CoV-2, middle east respiratory syndrome virus (MERS-CoV), alpha coronavirus 229E, alpha coronavirus NL63, beta coronavirus OC43, and beta coronavirus HKU1. In accordance with any of these embodiments, the PIKfyve inhibitor may be selected from the group consisting of apilimod, APY0201, YM201636, and pharmaceutically acceptable salts thereof. In accordance with any of these embodiments, the serine protease inhibitor may be selected from the group consisting of actinonin, amiloride, apixaban, aprotinin, argatroban, aspartame, atropine, batimastat, betrixaban, bromhixine, bupivacaine, camostat, cefixime, creatinine, dabigatran, dansyl-D-phenylalanine, darapladib, darexaban, dizocilpine, doxorubicin, doxycycline, ecopladib, edoxaban, efipladib, epichlortetracycline, epidemeclocycline, eribaxaban, gabexate, ginsenoside, giripladib, ilomastat, letaxaban, leupeptin, luteolin, marimastat, nafamostat, neostigmine, noradrenaline, otamixaban, phosphoramidon, prinomastat, rivaroxaban, sepimostat, sivelestat, suramin, tanomastat, thiorphan, tranexamic acid, trocade, tropicamide, varespladib, variabilin, and ximelagatran. In accordance with any of these embodiments, the serine protease inhibitor may be selected from aprotinin, bromhixine, camostat, gabexate, nafamostat, otamixaban, and sepimostat.

In accordance with any of the embodiments disclosed here, the coronavirus infection may be caused by a coronavirus selected from the group consisting of severe acute respiratory system virus (SARS-CoV), SARS-CoV-2, middle east respiratory syndrome virus (MERS-CoV), alpha coronavirus 229E, alpha coronavirus NL63, beta coronavirus OC43, and beta coronavirus HKU1, preferably SARS-CoV-2; the PIKfyve inhibitor may be selected from the group consisting of apilimod, APY0201, YM201636, and pharmaceutically acceptable salts thereof, preferably apilimod; and the serine protease inhibitor may be an inhibitor of TMPRSS2 selected from the group consisting of aprotinin, bromhixine, camostat, marimastat, nafamostat, phosphoramidon, sepimostat, and tannic acid; or in certain embodiments selected from camostat or nafamostat.

In certain specific embodiments of the compositions and methods described here, the coronavirus infection is caused by SARS-CoV-2, the PIKfyve inhibitor is apilimod, or a pharmaceutically acceptable salt thereof, and the serine protease inhibitor is an inhibitor of TMPRSS2, preferably selected from aprotinin, bromhixine, camostat, marimastat, nafamostat, phosphoramidon, sepimostat, and tannic acid, and most preferably camostat and nafamostat, and pharmaceutically acceptable salts thereof.

Apilimod is a selective inhibitor of PIKfyve (Cai et al. 2013 Chem. & Biol. 20:912-921). Based upon its ability to inhibit IL-12/23 production, apilimod has been suggested as useful for treating inflammatory and autoimmune diseases such as rheumatoid arthritis, sepsis, Crohn's disease, multiple sclerosis, psoriasis, or insulin dependent diabetes mellitus, and in cancers where these cytokines were believed to play a pro-proliferative role.

As used herein, the term “apilimod” refers to apilimod free base having the structure shown in Formula I:

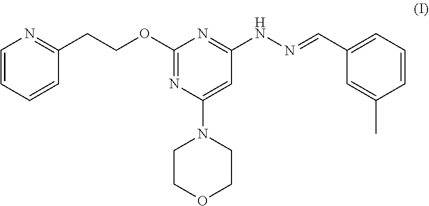 [SMILES: Cc1cccc(/C=N/Nc2cc(N3CCOCC3)nc(OCCc3ccccn3)n2)c1]

The chemical name of apilimod is 2-[2-Pyridin-2-yl)-ethoxy]-4-N′-(3-methyl-benzilidene)-hydrazino]-6-(morpholin-4-yl)-pyrimidine (IUPAC name: (E)-4-(6-(2-(3-methylbenzylidene)hydrazinyl)-2-(2-(pyridin-2-yl)ethoxy)pyrimidin-4-yl)morpholine), and the CAS number is 541550-19-0. Apilimod can be prepared, for example, according to the methods described in U.S. Pat. Nos. 7,923,557, and 7,863,270, and WO 2006/128129.

In embodiments, the apilimod may be administered in combination with at least one additional PIKfyve inhibitor selected from APY0201 and YM-201636. The chemical name of APY0201 is (E)-4-(5-(2-(3-methylbenzylidine)hydrazinlyl)-2-(pyridine-4-yl)pyrazolol[1,5-a]pyrimidin-7-yl)morpholine. APY0201 is a selective PIKfyve inhibitor (Hayakawa et al. (2014) Bioorg. Med. Chem. 22:3021-29). APY0201 directly interacts with the ATP-binding site of PIKfyve kinase, which leads to suppression of PI(3,5)P₂ synthesis, which in turn suppresses the production of IL-12/23. The chemical name for YM201636 is 6-amino-N-(3-(4-morpholinopyrido[3′,2′:4,5]furo[3,2-d]pyrimidin-2-yl)phenyl)nicotinamide (CAS number is 371942-69-7). YM201636 is a selective inhibitor of PIKfyve (Jefferies et al. EMBO rep. (2008) 9:164-170). It reversibly impairs endosomal trafficking in NIH3T3 cells, mimicking the effect produced by depleting PIKfyve with siRNA. YM201636 also blocks retroviral exit by budding from cells, apparently by interfering with the endosomal sorting complex required for transport (ESCRT) machinery. In adipocytes, YM-201636 also inhibits basal and insulin-activated 2-deoxyglucose uptake (IC₅₀=54 nM).

Camostat is a serine protease inhibitor whose targets include TMPRS22. As used herein, the term “camostat” refers to camostat free base having the structure shown in Formula II:

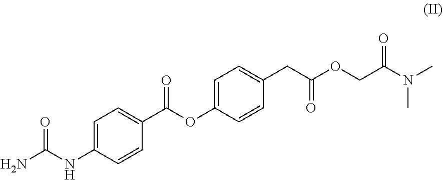 [SMILES: CN(C)C(=O)COC(=O)Cc1ccc(OC(=O)c2ccc(NC(N)=O)cc2)cc1]

The chemical name of camostat is [4-[2-[2-(dimethylamino)-2-oxoethoxy]-2-oxoethyl]phenyl]4-(diaminomethylideneamino)benzoate, and the CAS number is 59721-28-7. Molecular formula for camostat is C₂₀H₂₂N₄O₅ and molecular weight for camostat is 398.4 g/mol.

Nafamostat is a serine protease inhibitor whose targets include TMPRS22. As used herein, the term “nafamostat” refers to nafamostat free base having the structure shown in Formula III:

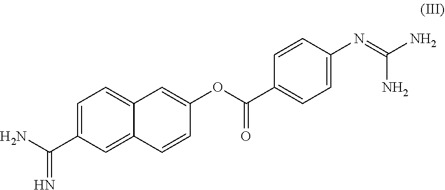 [SMILES: N=C(N)c1ccc2cc(OC(=O)c3ccc(N=C(N)N)cc3)ccc2c1]

The chemical name of nafamostat is (6-carbamimidoylnaphthalen-2-yl) 4-(diaminomethylideneamino)benzoate, and the CAS number is 81525-10-2. Molecular formula for camostat is C₁₉H₁₇N₅O₂ and molecular weight for nafamostat is 347 g/mol.

As used herein, the term “pharmaceutically acceptable salt,” is a salt formed from, for example, an acid and a basic group of a compound. Illustrative salts include, but are not limited to, sulfate, citrate, acetate, oxalate, chloride, bromide, iodide, nitrate, bisulfate, phosphate, acid phosphate, isonicotinate, L-lactate, D-lactate, DL-lactate, salicylate, acid citrate, L-tartrate, D-tartrate, DL-tartrate, oleate, tannate, pantothenate, bitartrate, ascorbate, succinate, maleate, besylate, gentisinate, fumarate, gluconate, glucaronate, saccharate, formate, benzoate, glutamate, methanesulfonate (mesylate), ethanesulfonate, benzenesulfonate, p-toluenesulfonate, and pamoate (e.g., 1,1′-methylene-bis-(2-hydroxy-3-naphthoate)) salts. In embodiments, the pharmaceutically acceptable salt form of apilimod is a methanesulfonate salt form, alternatively referred to as a mesylate salt.

The term “pharmaceutically acceptable salt” also refers to a salt prepared from a compound having an acidic functional group, such as a carboxylic acid functional group, and a pharmaceutically acceptable inorganic or organic base.

The term “pharmaceutically acceptable salt” also refers to a salt prepared from a compound having a basic functional group, such as an amino functional group, and a pharmaceutically acceptable inorganic or organic acid.

In embodiments, the salts include disalts in which the Bronsted acid is selected from a group consisting of hydrochloric acid, hydrobromic acid, nitric acid, sulfuric acid, hydrobromic acid, hydroiodic acid, perchloric acid, methanesulfonic acid, phorphoric acid, alkylsulfonic acids, arylsulfonic acids, halogenated alkylsulfonic acids, halogenated arylsulfonic acids, halogenated alkylsulfonic acids, halogenated acetic acids, picric acid, oxalic acid, citric acid, formic acid, ascorbic acid, benzoic acid and other salts possessing sufficient acidity to form a crystalline disalt of apilimod. In an embodiment, the salt form of apilimod is a dimesylate.

The salts of the compounds described herein can be synthesized from the parent compound by conventional chemical methods such as methods described in Pharmaceutical Salts: Properties, Selection, and Use, P. Hemrich Stalil (Editor), Camille G. Wermuth (Editor), ISBN: 3-90639-026-8, August 2002. Generally, such salts can be prepared by reacting the parent compound with the appropriate acid in water or in an organic solvent, or in a mixture of the two.

One salt form of a compound described herein can be converted to the free base and optionally to another salt form by methods well known to the skilled person. For example, the free base can be formed by passing the salt solution through a column containing an amine stationary phase (e.g. a Strata-NH₂ column). Alternatively, a solution of the salt in water can be treated with sodium bicarbonate to decompose the salt and precipitate out the free base. The free base may then be combined with another acid using routine methods.

In embodiments of the compositions and methods described here, a pharmaceutical composition comprises a prophylactically or therapeutically effective amount of a PIKfyve inhibitor and a serine protease inhibitor. A therapeutically effective amount is the amount effective to treat a subject or patient. As used herein, the terms “treatment”, “treating” and “treat” describe the management and care of a subject or patient for the purpose of combating a viral disease and includes, alleviating, reducing, or eliminating one or more symptoms or complications of the viral disease. A prophylactically effective amount is the amount effective to prevent viral disease in a subject or patient. As used herein, the terms “prevention,” “preventing” and “prevent” describe reducing or eliminating the onset of symptoms or complications of the viral disease, or reducing or eliminating development of viral disease in a subject or patient exposed to the virus. The term “effective amount” in the context of a prophylactically or therapeutically effective amount refers to an amount sufficient to prevent or treat a viral disease. In embodiments, the effective amount is an amount effective to achieve one or more of the following: inhibit cellular activity of PIKfyve or TMPRSS2; substantially prevent viral entry into a subject's cells; reduce the amount of viral particles which gain entry to a subject's cells; reduce viral replication within the subject's cells; ameliorate one or more symptoms associated with viral infection of the subject; or reduce the severity of one or more symptoms associated with viral infection of the subject.

In embodiments, a therapeutically effective amount is an amount sufficient to reduce viral load. In embodiments, the viral load is reduced by 5% or greater, 10% or greater, 20% or greater, 30% or greater, 40% or greater, 50% or greater, or 75% or greater. In embodiments, the viral load is reduced by at least 0.5 log unit, at least 1 log unit, at least 2 log units, at least 3 log units, at least 4 log units, at least 10 log units, at least 15 log units, or by at least 20 log units.

In embodiments, a prophylactically effective amount is an amount sufficient to substantially reduce the infectivity or cytotoxicity of a virus; or substantially reduce the severity of, or eliminate, one or more symptoms or complications associated with viral infection in a subject exposed to the virus.

In embodiments, the prophylactically or therapeutically effective amount of apilimod when administered to a human subject in combination with a serine protease inhibitor as described here is from about 1 to 250 mg/day, from about 10 to 250 mg/day, from about 20 to 250 mg/day, from about 30 to 250 mg/day, from about 40 to 250 mg/day, from about 50 to 250 mg/day, from about 60 to 250 mg/day, or from about 70 to 250 mg/day. In embodiments, the prophylactically or therapeutically effective amount of apilimod when administered to a human subject in combination with a serine protease inhibitor as described here is from about 1 to 200 mg/day, from about 10 to 200 mg/day, from about 20 to 200 mg/day, from about 30 to 200 mg/day, from about 40 to 200 mg/day, from about 50 to 200 mg/day, from about 60 to 200 mg/day, or from about 70 to 200 mg/day. In embodiments, the prophylactically or therapeutically effective amount of apilimod when administered to a human subject in combination with a serine protease inhibitor as described here is from about 1 to 150 mg/day, from about 10 to 150 mg/day, from about 20 to 150 mg/day, from about 30 to 150 mg/day, from about 40 to 150 mg/day, from about 50 to 150 mg/day, from about 60 to 150 mg/day, or from about 70 to 150 mg/day. In embodiments, the prophylactically or therapeutically effective amount of apilimod when administered to a human subject in combination with a serine protease inhibitor as described here is from about 1 to 100 mg/day, from about 10 to 100 mg/day, from about 20 to 100 mg/day, from about 30 to 100 mg/day, from about 40 to 100 mg/day, from about 50 to 100 mg/day, from about 60 to 100 mg/day, or from about 70 to 100 mg/day.

In embodiments, the prophylactically or therapeutically effective amount of apilimod is from 150-250 mg/day or from 75-125 mg twice daily. In embodiments, the prophylactically or therapeutically effective amount of apilimod is an amount effective to achieve a plasma concentration of apilimod in the subject in the range of from 50 to 1000 nM.

In embodiments, the prophylactically or therapeutically effective amount of camostat mesylate in humans is from about 70 to 2000 mg/day, from about 70 to 1000 mg/day, from about 70 to 800 mg/day, from about 70 to 600 mg/day, from about 70 to 300 mg/day, of from about 70 to 200 mg/day. In embodiments, the prophylactically or therapeutically effective amount of camostat mesylate is an amount effective to achieve a plasma concentration of its active metabolite 4-(4-guanidinobenzoyloxy) phenylacetate in the subject after a single dose in the range of from 10-500 ng/ml.

In embodiments, the prophylactically or therapeutically effective amount of camostat mesylate when administered to a human subject in combination with apilimod is from about 70 to 500 mg/day, from about 70 to 400 mg/day, from about 70 to 300 mg/day, or from about 70 to 200 mg/day.

### Methods of Treatment

The present invention provides methods for the prevention or treatment of viral disease, preferably one caused by a coronavirus, in a subject in need thereof, the methods comprising administering to the subject a prophylactically or therapeutically effective amount of a PIKfyve inhibitor in combination with a serine protease inhibitor, as described herein. In embodiments, the PIKfyve inhibitor is selected from apilimod, APY0201, YM-201636, or a pharmaceutically acceptable salts thereof, and combinations thereof. In embodiments, the serine protease inhibitor is selected from the group consisting of actinonin, amiloride, apixaban, aprotinin, argatroban, aspartame, atropine, batimastat, betrixaban, bromhixine, bupivacaine, camostat, cefixime, creatinine, dabigatran, dansyl-D-phenylalanine, darapladib, darexaban, dizocilpine, doxorubicin, doxycycline, ecopladib, edoxaban, efipladib, epichlortetracycline, epidemeclocycline, eribaxaban, gabexate, ginsenoside, giripladib, ilomastat, letaxaban, leupeptin, luteolin, marimastat, nafamostat, neostigmine, noradrenaline, otamixaban, phosphoramidon, prinomastat, rivaroxaban, sepimostat, sivelestat, suramin, tanomastat, thiorphan, tranexamic acid, trocade, tropicamide, varespladib, variabilin, and ximelagatran. In embodiments, the serine protease inhibitor is selected from the group consisting of aprotinin, bromhixine, camostat, gabexate, nafamostat, otamixaban, and sepimostat.

In further embodiments, the invention provides methods for the prevention or treatment of viral disease, preferably one caused by a coronavirus, in a subject in need thereof, the methods comprising administering to the subject a prophylactically or therapeutically effective amount of a PIKfyve inhibitor in combination with a serine protease inhibitor, wherein the PIKfyve inhibitor is selected from apilimod, APY0201, YM-201636, or a pharmaceutically acceptable salts thereof, and combinations thereof, preferably apilimod or a combination comprising apilimod, and the serine protease inhibitor is an inhibitor of TMPRSS2 selected from the group consisting of aprotinin, bromhixine, camostat, marimastat, nafamostat, phosphoramidon, sepimostat, and tannic acid, preferably selected from camostat and nafamostat. In certain specific embodiments of the methods described here, the PIKfyve inhibitor is apilimod, or a pharmaceutically acceptable salt thereof, and the serine protease inhibitor is an inhibitor of TMPRSS2, preferably selected from camostat and nafamostat, and pharmaceutically acceptable salts thereof, preferably a mesylate salt.

In some embodiments, the methods comprise administering the PIKfyve inhibitor in combination with a serine protease inhibitor according to a specified dosing schedule or therapeutic regimen. For example, the PIKfyve inhibitor and the serine protease inhibitor can be administered in any sequential steps once daily, twice, thrice, or four times daily.

In the context of combination therapy, a PIKfyve inhibitor and a serine protease inhibitor described herein may be administered in separate dosage forms, or in the same dosage form. Where the inhibitors are administered in separate dosage forms, they may be administered at the same time, or at different times.

In accordance with the methods described herein, a “subject in need of” is a subject having a coronavirus infection, or a subject having an increased risk of developing a coronavirus infection. The subject in need thereof can be one that is “non-responsive” or “refractory” to a currently available therapy for the viral disease. In this context, the terms “non-responsive” and “refractory” refer to the subject's response to therapy as not clinically adequate to relieve one or more symptoms associated with the viral infection. In one aspect of the methods described here, the subject in need thereof is a subject having a viral disease caused by a coronavirus who is refractory to standard therapy.

A “subject” includes a mammal. The mammal can be e.g., any mammal, e.g., a human, primate, mouse, rat, dog, cat, cow, horse, goat, camel, sheep or a pig. Preferably, the mammal is a human. The term “patient” refers to a human subject.

As used herein, “combination therapy” or “co-therapy” includes the administration of a therapeutically effective amount of a PIKfyve inhibitor, preferably apilimod, in combination with a serine protease inhibitor as described herein, as part of a specific treatment regimen intended to provide a beneficial effect from the co-action of the active agents in the regimen. “Combination therapy” is not intended to encompass the administration of two or more therapeutic compounds as part of separate monotherapy regimens that incidentally and arbitrarily result in a beneficial effect that was not intended or predicted.

Thus, the invention also provides methods of treating a subject for a viral disease or viral infection (the terms “viral disease” and “viral infection” are used interchangeably herein) using a combination therapy comprising a PIKfyve inhibitor, preferably apilimod, in combination with a serine protease inhibitor as described herein, in an antiviral regimen for the treatment of the viral disease. In embodiments, the antiviral regimen may comprise one or more antiviral agents, in addition to the PIKfyve inhibitor and the serine protease inhibitor. In some embodiments, the antiviral agent is selected from an antiviral vaccine, a nucleotide analogue inhibitor of a viral polymerase, a cytokine (e.g., an interferon), an immunoglobulin, a JAK inhibitor (e.g., baracitinib) and combinations thereof. In embodiments, the antiviral agent is selected from an inhibitor of one or more of NPCI, VPSII, VPSI6, VPSI8, Vacuolar Protein Sorting 33 Homolog A (VPS33A), Vacuolar Protein Sorting 39 Homolog (VPS39), Vacuolar Protein Sorting 41 Homolog (VPS41), BLOCISI, BLOCIS2, N-Acetylglucosamine-1-Phosphate Transferase, Alpha And Beta Subunits (GNPT-AB), Phosphoinositide Kinase, FYVE Finger Containing (PIKFYVE), ARGHGAP23, coat protein complex 1 (COPI), coat protein complex II (COPII), Mannose-6-phosphate receptor binding protein 1 (TIP47), Interleukin 12 (IL-12 or P40), Rab GTP-binding proteins (e.g., Rab9), clathrin, activator protein 1 (AP1), adaptor protein 3 (AP3), vesicle soluble N-ethylmaleimide-sensitive factor attachment protein receptor (v-SNARE), target soluble N-ethylmaleimide-sensitive factor attachment protein receptor (t-SNARE), ADP-ribosylation factor 1 (ARFs), Ras GTP-ases, and a combinations thereof.

Further non-limiting examples of antiviral agents that may be used in combination therapy comprising a PIKfyve inhibitor, preferably apilimod, in combination with a serine protease inhibitor as described herein, in an antiviral regimen for the treatment of the viral disease include acemannan; acyclovir; acyclovir sodium; adefovir; alovudine; alvircept sudotox; amantadine hydrochloride; aranotin; arildone; atevirdine mesylate; avridine; chloroquine; cidofovir; cipamfylline; cytarabine hydrochloride; delavirdine mesylate; desciclovir; didanosine; disoxaril; edoxudine; enviradene; enviroxime; famciclovir; famotine hydrochloride; fiacitabine; fialuridine; fosarilate; foscarnet sodium; fosfonet sodium; ganciclovir; ganciclovir sodium; idoxuridine; kethoxal; lamivudine; lobucavir; memotine hydrochloride; methisazone; nevirapine; penciclovir; pirodavir; ribavirin; remdesivir; rimantadine hydrochloride; saquinavir mesylate; somantadine hydrochloride; sorivudine; statolon; stavudine; tilorone hydrochloride; trifluridine; valacyclovir hydrochloride; vidarabine; vidarabine phosphate; vidarabine sodium phosphate; viroxime; zalcitabine; zidovudine; and zinviroxime.

In embodiments, the antiviral regimen may comprise an antibody or a combination of antibodies, preferably human or humanized antibodies, but chimeric (e.g., mouse-human chimeras) antibodies are also acceptable. In embodiments, the antiviral regimen may comprise a small interfering RNA (siRNA) or a combination of siRNA molecules. In embodiments, the antiviral regimen may comprise an antibody, siRNA, or small organic molecule targeted to inhibit the activity of one or more viral proteins, for example a polymerase, a membrane-associated protein, a polymerase complex protein, a protease, a helicase, an envelope, a nucleocapsid, a spike glycoprotein, a viral structural, or a viral accessory protein. In embodiments, the siRNA or combination of siRNAs, the antibody or combination of antibodies, the protein or combination of proteins targets one or more host proteins. In embodiments, the antiviral regimen may comprise an antibody, siRNA, or small organic molecule targeted to inhibit the activity of one or more host proteins, for example, interferon, a cell surface receptor, a protease, or a protein involved in endosomal trafficking or acidification. In embodiments, the antibody, recombinant protein, or siRNA.

In embodiments, the antiviral regimen may comprise one or more of an interferon, remdesivir, azithromycin, hydroxychloroquine and chloroquine. In embodiments, the antiviral regimen may comprise an anti-inflammatory agent. In embodiments, the anti-inflammatory agent is selected from tocilizumab and sarilumab.

In certain embodiments, the at least one PIKfyve inhibitor is provided in a single dosage form in combination with a serine protease inhibitor as described herein. In embodiments, the at least one PIKfyve inhibitor is provided in a separate dosage form from a serine protease inhibitor as described herein. Separate dosage forms are desirable, for example, in the context of a combination therapy in which the therapeutic regimen calls for dose modification, administration of different therapeutic agents at different frequencies or under different conditions, or via different routes.

In embodiments, administration of the at least one PIKfyve inhibitor and serine protease inhibitor as described herein is accomplished via an oral dosage form suitable for oral administration. In another embodiment, administration is by an indwelling catheter, a pump, such as an osmotic minipump, or a sustained release composition that is, for example, implanted in the subject, or by intravenous administration.

### Pharmaceutical Compositions and Formulations

The disclosure provides pharmaceutical compositions comprising an effective amount of a PIKfyve inhibitor, preferably apilimod, a serine protease inhibitor, and at least one pharmaceutically acceptable excipient or carrier. In embodiments, the composition is an oral dosage form selected from a capsule or tablet. In embodiments, the composition is in the form of an orally dissolving tablet.

In embodiments, the PIKfyve inhibitor is selected from apilimod, APY0201, YM-201636, and pharmaceutically acceptable salts thereof. In embodiments, the PIKfyve inhibitor is apilimod, or a pharmaceutically acceptable salt thereof.

In embodiments, the serine protease inhibitor is selected from the group consisting of actinonin, amiloride, apixaban, aprotinin, argatroban, aspartame, atropine, batimastat, betrixaban, bromhixine, bupivacaine, camostat, cefixime, creatinine, dabigatran, dansyl-D-phenylalanine, darapladib, darexaban, dizocilpine, doxorubicin, doxycycline, ccopladib, edoxaban, efipladib, epichlortetracycline, epidemeclocycline, eribaxaban, gabexate, ginsenoside, giripladib, ilomastat, letaxaban, leupeptin, lutcolin, marimastat, nafamostat, neostigmine, noradrenaline, otamixaban, phosphoramidon, prinomastat, rivaroxaban, sepimostat, sivelestat, suramin, tanomastat, thiorphan, tranexamic acid, trocade, tropicamide, varespladib, variabilin, and ximelagatran. In embodiments, the serine protease inhibitor is selected from the group consisting of aprotinin, bromhixine, camostat, gabexate, nafamostat, otamixaban, and sepimostat.

In embodiments, the serine protease inhibitor is an inhibitor of transmembrane protease, serine 2 (TMPRSS2). In embodiments, the TMPRSS2 inhibitor is selected from the group consisting of aprotinin, bromhixine, camostat, marimastat, nafamostat, phosphoramidon, sepimostat, and tannic acid. In embodiments, the serine protease inhibitor is a TMPRSS2 inhibitor selected from the group consisting of camostat and nafamostat.

The term “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof.

A pharmaceutical composition can be provided in bulk or in dosage unit form. It is especially advantageous to formulate pharmaceutical compositions in dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved. A dosage unit form can be an ampoule, a vial, a suppository, a dragee, a tablet, a capsule, an IV bag, or a single pump on an aerosol inhaler.

In therapeutic applications, the dosages vary depending on the agent, the age, weight, and clinical condition of the recipient patient, and the experience and judgment of the clinician or practitioner administering the therapy, among other factors affecting the selected dosage. Generally, the dose should be a therapeutically effective amount. Dosages can be provided in mg/kg/day units of measurement (which dose may be adjusted for the patient's weight in kg, body surface area in m², and age in years). Exemplary doses and dosages regimens for the compositions in methods of treating viral infections are described above.

A dose may be provided in unit dosage form. For example, the unit dosage form can comprise 25-150 mg apilimod free base (or equivalent amount of a pharmaceutically acceptable salt form of apilimod) and from 100-1000 mg camostat mesylate.

The pharmaceutical compositions can take any suitable form (e.g., liquids, aerosols, solutions, inhalants, mists, sprays; or solids, powders, ointments, pastes, creams, lotions, gels, patches and the like) for administration by any desired route (e.g., pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the invention may be in the form of an aqueous solution or powder for aerosol administration by inhalation or insufflation (either through the mouth or the nose), in the form of a tablet or capsule for oral administration; in the form of a sterile aqueous solution or dispersion suitable for administration by either direct injection or by addition to sterile infusion fluids for intravenous infusion; or in the form of a lotion, cream, foam, patch, suspension, solution, or suppository for transdermal or transmucosal administration.

A pharmaceutical composition can be in the form of an orally acceptable dosage form including, but not limited to, capsules, tablets, orally dissolving tablets, buccal forms, troches, lozenges, and oral liquids in the form of emulsions, aqueous suspensions, dispersions or solutions. Capsules may contain mixtures of a compound of the present invention with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. In the case of tablets for oral use, carriers which are commonly used include lactose and corn starch. Lubricating agents, such as magnesium stearate, can also be added. For oral administration in a capsule form, useful diluents include lactose and dried corn starch. When aqueous suspensions and/or emulsions are administered orally, the compound of the present invention may be suspended or dissolved in an oily phase is combined with emulsifying and/or suspending agents. If desired, certain sweetening and/or flavoring and/or coloring agents may be added.

A pharmaceutical composition can be in the form of a tablet. The tablet can comprise a unit dosage of a compound of the present invention together with an inert diluent or carrier such as a sugar or sugar alcohol, for example lactose, sucrose, sorbitol or mannitol. The tablet can further comprise a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. The tablet can further comprise binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures.

The tablet can be a coated tablet. The coating can be a protective film coating (e.g. a wax or varnish) or a coating designed to control the release of the active agent, for example a delayed release (release of the active after a predetermined lag time following ingestion) or release at a particular location in the gastrointestinal tract. The latter can be achieved, for example, using enteric film coatings such as those sold under the brand name Eudragit®.

Tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine.

A pharmaceutical composition can be in the form of a hard or soft gelatin capsule. In accordance with this formulation, the compound of the present invention may be in a solid, semi-solid, or liquid form.

A pharmaceutical composition can be in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

A pharmaceutical composition can be in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions of the compound of the present invention as a free base or pharmacologically acceptable salt can be prepared in water suitably mixed with a surfactant. Examples of suitable surfactants are given below. Dispersions can also be prepared, for example, in glycerol, liquid polyethylene glycols and mixtures of the same in oils.

The pharmaceutical compositions for use in the methods of the present invention can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants, the HLB values can differ by as much as about 8 HLB units, depending upon the empirical method chosen to determine the HLB value.

Among the surfactants for use in the compositions of the invention are polyethylene glycol (PEG)-fatty acids and PEG-fatty acid mono and diesters, PEG glycerol esters, alcohol-oil transesterification products, polyglyceryl fatty acids, propylene glycol fatty acid esters, sterol and sterol derivatives, polyethylene glycol sorbitan fatty acid esters, polyethylene glycol alkyl ethers, sugar and its derivatives, polyethylene glycol alkyl phenols, polyoxyethylene-polyoxypropylene (POE-POP) block copolymers, sorbitan fatty acid esters, ionic surfactants, fat-soluble vitamins and their salts, water-soluble vitamins and their amphiphilic derivatives, amino acids and their salts, and organic acids and their esters and anhydrides.

The present invention also provides packaging and kits comprising pharmaceutical compositions for use in the methods of the present invention. The kit can comprise one or more containers selected from the group consisting of a bottle, a vial, an ampoule, a blister pack, and a syringe. The kit can further include one or more of instructions for use in treating and/or preventing a disease, condition or disorder of the present invention, one or more syringes, one or more applicators, or a sterile solution suitable for reconstituting a pharmaceutical composition of the present invention.

All percentages and ratios used herein, unless otherwise indicated, are by weight. Other features and advantages of the present invention are apparent from the different examples. The following examples illustrate different components and methodology useful in practicing the present invention. The examples do not limit the claimed invention. Based on the present disclosure the skilled artisan can identify and employ other components and methodology useful for practicing the present invention.

### EXAMPLES

### Example 1

An in vitro antiviral activity assay, cytopathic effect reduction, was used to evaluate the activity of apilimod, cither alone or in combination with a serine protease inhibitor, against coronavirus infection. Briefly, Caco-2 cells were seeded at optimal density for growth into 96-well plates and placed into a humidified incubator (37° C., 5% CO₂) to adhere overnight. The following day, cells were treated for two hours with either single agent (apilimod or camostat) or a combination of the two agents. Cells were then infected with SARS-CoV-2 (USA-WA1/2020) at an MOI of 0.02. After 72 hr the supernatant was collected, and viral titer determined by endpoint dilution and cytopathic effect in Vero cells was determined by measuring cell viability using a colorimetric assay (Neutral Red).

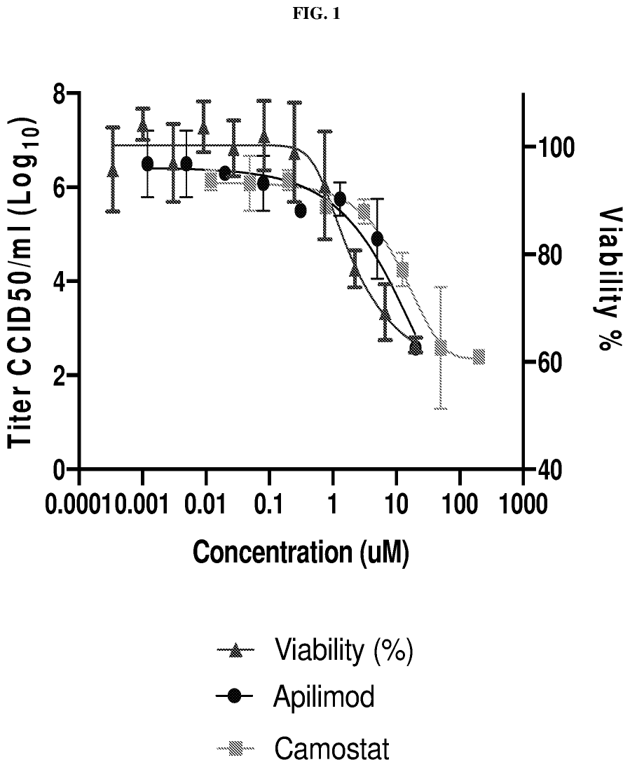

The single agent treatments consisted of 6 or 7 different concentrations obtained by serial dilution. The concentrations were chosen to provide a range above and below the IC50 value of the single agent. Table 1 shows the concentration ranges used for each of the agents tested. Drug cytotoxicity on the Caco-2 cells was measured in parallel via neutral red assay. No combination exhibited greater than a 10% average reduction in viability compared to DMSO controls. The percent viral infection was then calculated for each single agent concentration (Table 2) or combination (Table 3), as compared to DMSO controls. FIG. 1 shows dose response curves for each single agent treatment with apilimod or camostat, confirming the anti-viral functions of these two agents. Synergistic activity was assessed using the combination index (CI) values calculated according to the Chou-Talalay method as described in Chou T-C, (2010) Cancer Res. 70(2), 440-446. Using this method, a CI value is calculated based on the effects of each drug alone (single agent treatment) and the effects of each drug combination, across all combinations of both agents tested (a matrix of 36 or 49 combinations). Two independent experiments were performed with 2 replicates per experiment.

TABLE 1
Agents and concentration ranges used for
assessment of synergistic activity.

| Target | Agent | Concentration range (fold dilution) |
|---|---|---|
| PIKfyve | Apilimod | 31.25-2000 nM (2-fold) |
| Serine proteases | Camostat | 70-45000 nM (2-fold) |
| including ACE2 | | |

TABLE 2
Normalized SARS-CoV-2 infection for single agent
(apilimod or camostat) in Caco-2 cells.

| Apilimod | | | | | |
|---|---|---|---|---|---|
| (nM) | Percent | Percent | Camostat | Percent | Percent |
| (single | Infection | Infection | (nM) (single | Infection | Infection |
| agent) | (Exp 1) | (Exp 2) | agent) | (Exp 1) | (Exp 2) |
| 2000 | 0.591 | 0.297 | 4500 | 0.435 | 0.196 |
| 1000 | 4.524 | 2.968 | 2250 | 4.345 | 0.373 |
| 500 | 12.267 | 18.730 | 1125 | 9.363 | 2.968 |
| 250 | 11.935 | 29.685 | 563 | 43.450 | 11.510 |
| 125 | 35.618 | 37.259 | 281 | 59.680 | 37.259 |
| 62.5 | 75.308 | 53.914 | 141 | 75.308 | 74.342 |
| 31.25 | 93.632 | 74.342 | 70 | 119.354 | 148.332 |

TABLE 3
Normalized percent SARS-CoV-2 infection for each of the
49 combination treatments of apilimod in Caco-2 cells.
Experiment 1

| | | | | | | | | |
|---|---|---|---|---|---|---|---|---|
| Apilimod | 2000 | 0.045 | 0.015 | 0.015 | 0.015 | 0.015 | 0.015 | 0.015 |
| (nM) | 1000 | 4.345 | 1.227 | 1.227 | 0.119 | 0.015 | 0.015 | 0.015 |
| | 500 | 9.363 | 5.908 | 3.573 | 0.218 | 0.023 | 0.015 | 0.015 |
| | 250 | 9.363 | 12.267 | 6.356 | 0.452 | 0.123 | 0.015 | 0.015 |
| | 125 | 14.840 | 29.609 | 77.402 | 3.562 | 1.194 | 0.022 | 0.015 |
| | 62.5 | 93.632 | 93.632 | 77.402 | 9.363 | 1.227 | 0.774 | 0.022 |
| | 31.25 | 93.632 | 77.402 | 59.680 | 21.777 | 9.363 | 1.484 | 0.227 |
| | | 70 | 141 | 282 | 563 | 1125 | 2250 | 4500 |

Camostat (nM)

Experiment 2

| | | | | | | | | |
|---|---|---|---|---|---|---|---|---|
| Apilimod | 2000 | 0.005 | 0.004 | 0.004 | 0.004 | 0.004 | 0.004 | 0.004 |
| (nM) | 1000 | 1.483 | 0.311 | 0.311 | 0.024 | 0.004 | 0.004 | 0.004 |
| | 500 | 3.108 | 0.539 | 0.463 | 0.037 | 0.005 | 0.004 | 0.004 |
| | 250 | 3.726 | 3.726 | 2.491 | 0.196 | 0.046 | 0.006 | 0.004 |
| | 125 | 11.510 | 14.833 | 12.611 | 0.373 | 0.373 | 0.012 | 0.004 |
| | 62.5 | 37.259 | 37.259 | 29.685 | 3.108 | 0.463 | 0.196 | 0.012 |
| | 31.25 | 57.687 | 37.259 | 31.084 | 5.769 | 1.961 | 0.311 | 0.030 |
| | | 70 | 141 | 282 | 563 | 1125 | 2250 | 4500 |

Camostat (nM)

Table 4 below provides a summary of the average CI values obtained from the two independent experiments illustrated in Table 3. Using this method, combinations with CI values>1 are considered antagonistic, CI values=1 are considered additive, and CI values<1 are considered synergistic.

TABLE 4
Combination Index (CI) values for the 49 (7 × 7)
combination treatments of apilimod and camostat.

| | | | | | | | | |
|---|---|---|---|---|---|---|---|---|
| Apilimod | 2000 | 0.08 | 0.05 | 0.05 | 0.06 | 0.09 | 0.14 | 0.25 |
| (nM) | 1000 | 0.8 | 0.35 | 0.39 | 0.11 | 0.07 | 0.13 | 0.22 |
| | 500 | 0.71 | 0.48 | 0.42 | 0.22 | 0.07 | 0.11 | 0.22 |
| | 250 | 0.4 | 1.05 | 0.45 | 0.14 | 0.13 | 0.11 | 0.21 |
| | 125 | 0.38 | 0.71 | 1.38 | 0.28 | 0.36 | 0.12 | 0.19 |
| | 62.5 | 1.35 | 1.46 | 1.57 | 0.54 | 0.73 | 0.5 | 0.23 |
| | 31.25 | 1.42 | 1.07 | 1.19 | 0.8 | 0.87 | 0.65 | 0.44 |
| | | 70 | 141 | 282 | 563 | 1125 | 2250 | 4500 |

Camostat (nM)

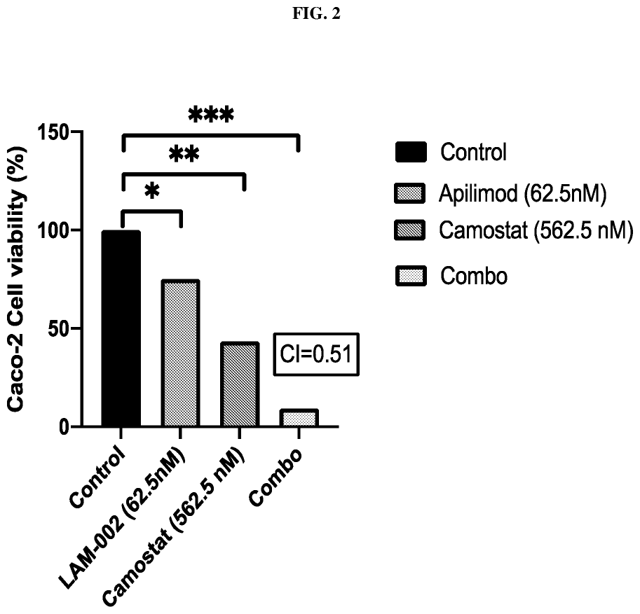

As shown in Table 4, a number of dose combination produced CI values<1, indicating synergy. These results show that apilimod is synergistic with the serine protease inhibitor camostat in preventing SARS-CoV-2 infection in Caco-2 cells. FIG. 2 illustrates the results of an exemplary synergistic combination of 62.5 nM apilimod and 562.5 nM camostat (rounded to 563 nM in the table) on SARS-CoV-2 infection.

1. A pharmaceutical composition comprising apilimod, or a pharmaceutically acceptable salt thereof, and a serine protease inhibitor.

2. The pharmaceutical composition of claim 1, wherein the apilimod is in the form of a free base.

3. The pharmaceutical composition of claim 1, wherein the apilimod is in the form of a salt and the pharmaceutically acceptable salt is a monosalt selected from the group consisting of chloride, fumarate, glycolate, D or L-lactate, maleate, malonate, mesylate, 1-hydroxy-2-naphthoate, phosphate, succinate, and L-tartrate.

4. The pharmaceutical composition of claim 1, wherein the apilimod is in the form of a salt and the pharmaceutically acceptable salt is a disalt selected from the group consisting of mesylate, chloride, and bromide.

5. The pharmaceutical composition of claim 1, wherein the apilimod is in the form of a dimesylate salt.

6. The pharmaceutical composition of claim 1, wherein the serine protease inhibitor is selected from the group consisting of actinonin, amiloride, apixaban, aprotinin, argatroban, aspartame, atropine, batimastat, betrixaban, bromhexine, bupivacaine, camostat, cefixime, creatinine, dabigatran, dansyl-D-pheylalanine, darapladib, darexaban, dizocilpine, doxorubicin, doxycycline, ecopladib, edoxaban, efipladib, epichlortetracycline, epidemeclocycline, eribaxaban, gabexate, ginsenoside, giripladib, ilomastat, letaxaban, leupeptin, luteolin, marimastat, nafamostat, neostigmine, noradrenaline, otamixaban, phosphoramidon, prinomastat, rivaroxaban, sepimostat, sivelestat, suramin, tanomastat, thiorphan, tranexamic acid, trocade, tropicamide, varespladib, variabilin, and ximelagatran.

7. The pharmaceutical composition of claim 6, wherein the serine protease inhibitor is selected from the group consisting of aprotinin, bromhexine, camostat, gabexate, nafamostat, otamixaban, and spimostat.

8. The pharmaceutical composition of claim 1, wherein the serine protease inhibitor is an inhibitor of transmembrane protease, serine 2 (TMPRSS2).

9. The pharmaceutical composition of claim 8, wherein the TMPRSS2 inhibitor is selected from the group consisting of aprotinin, bromhexine, camostat, marimastat, nafamostat, phosphoramidon, sepimostat, and tannic acid.

10. The pharmaceutical composition of claim 1, wherein the serine protease inhibitor is camostat or nafamostat.

11. The pharmaceutical composition of claim 1, wherein the composition is in the form of an oral dosage form or an intravenous dosage form.

12. A method for treating or preventing viral disease in a subject in need thereof, the method comprising administering to the subject a pharmaceutical composition comprising apilimod, or a pharmaceutically acceptable salt thereof, and a serine protease inhibitor.

13. The method of claim 12, wherein the apilimod is in the form of a free base.

14. The method of claim 12, wherein the apilimod is in the form of a salt and the pharmaceutically acceptable salt is a monosalt selected from the group consisting of chloride, fumarate, glycolate, D or L-lactate, maleate, malonate, mesylate, 1-hydroxy-2-naphthoate, phosphate, succinate, and L-tartrate.

15. The method of claim 12, wherein the apilimod is in the form of a salt and the pharmaceutically acceptable salt is a disalt selected from the group consisting of mesylate, chloride, and bromide.
